(19) United States
(12) Patent Application Publication
(10) Pub. No.: US 2015/0305797 A1
(43) Pub. Date: Oct. 29, 2015

(54) SURGICAL INSTRUMENT

(71) Applicant: Basel S. HASSOUN, Oklahoma City, OK (US)

(72) Inventor: Basel S. HASSOUN, Oklahoma City, OK (US)

(21) Appl. No.: 14/426,476

(22) PCT Filed: Sep. 7, 2012
(86) PCT No.: PCT/US12/54273

§ 371 Date: Mar. 6, 2015

(51) Int. Cl. A61B 18/08

(52) U.S. Cl. CPC A61B 18/085; A61B 2018/00595

(57) ABSTRACT

An improved surgical apparatus for use in endoscopic surgery having a user interface operatively coupled to an articulating tool assembly via an elongate tubular member. Movement at the articulating tool directly translates movement at the user interface thereby providing intuitive operation of the surgical apparatus. The apparatus is configured such that the range of motion of the articulating tool assembly emulates the range of motion of the human wrist. The improved apparatus is configured to provide precise and controlled movements such as to not endanger the life of the subject and minimizes any errant or unexpected movements or locking experienced in other surgical instruments.

### BACKGROUND

Minimally invasive surgery (MIS), such as laparoscopic surgery and thoracoscopic surgery are specialized types of surgery in the broader field of endoscopy. Laparoscopic surgery includes operations within the abdominal and pelvic cavities; thoracoscopic surgery includes operations within the thoracic cavity. Various tools and instruments are utilized during these procedures.

Such tools for MIS include robotic assisted instruments and various forms of hand-operated instruments. Unfortunately, robotic assisted instruments require extensive training, are expensive and bulky. Additionally, some hand-operated instruments are counter-intuitive, i.e. movement in the tool end is opposite from movement at the user interface or actuation end. For instance, when the operator moves the user interface right, the tool end moves left. Like the counter-intuitive instruments, intuitive hand-operated instruments have limited mobility and flexibility. Movements are more discrete, such as left, right, up, down, and do not provide transitional movement through all angular ranges. Thus, in order to obtain further articulation of the tool end, the user must physically reposition him/herself and/or the instrument.

All surgeries require precision and control, otherwise the patient's or subject's well-being is comprised. Some instruments do not provide sufficient routing and/or tension on cabling members and experience locking where the instrument is unable to return to its original neutral position. The locking of the instrument may also cause the tool end of the instrument to exhibit unexpected, unintentional, and/or errant movements, where the tool end either has a delayed movement based on input and/or the tool end unexpectedly releases itself from a locked position. In instruments where a solid pivot fastener is used, i.e. no central passageway through the center of the pivot point, cabling members are directed around the pivot point. In these instruments for example, moving the tip assembly (graspers, pincers etc.) down causes the tool end to also move left and the tip assembly to open as these cabling members experience tension due to the pulling on the down cable. Such errant movement is unacceptable in surgical procedures. These instruments may also suffer from “locking up” or becoming stuck in a position once actuated. For example, a cabling member will cross over the longitudinal axis of the instrument and become entangled with another cabling member and/or be unable to be able to return to its initial position or any other position as the requisite tension to move the component is not longer present.

### SUMMARY

The instrument described herein provides an intuitive hand-operated instrument capable of intricate movements with a range of motion comparable to that of a human wrist. The user provided with tactile feedback of the instrument's movements. The instrument described herein also provides the precision and control needed and expected by the operator in order to maintain the well-being of the patient. Further, the instrument described herein is a low cost, reliable, portable instrument that is safe for use in surgery, durable for repeated use, adaptable to the physical limitations of various procedures, and is easy to learn and use.

In one embodiment, an improved surgical instrument includes a user interface operatively coupled to an articulating tool assembly through an elongate tubular member having a distal end and a proximal end. The user interface is coupled to the proximal end of the elongate tubular member and the articulating tool assembly coupled to the distal end of the elongate tubular member. At least one cabling member extends through the elongate tubular member connecting the user interface and the articulating tool assembly such that movement of the user interface causes corresponding movement in the same direction of the user interface at the articulating tool assembly. The articulating tool assembly includes a grip assembly and a dual hinge member pivotally coupled together. The dual hinge member has a central passageway passing therethrough and includes at least one cable alignment surface. The user interface includes a ball and socket assembly. The ball and socket assembly has a socket with openings carrying a ball. The ball has protruding arms carrying a spring tension assembly projecting from the socket openings.

In another embodiment, an improved articulating tool assembly includes a tip assembly pivotally coupled to a dual hinge member. The dual hinge member has a central passageway passing therethrough and includes at least one cable alignment surface. The articulating tool assembly is configured to provide precise movements and prevent errant movements.

The objects, features, and advantages of the instrument will be readily apparent to those skilled in the art upon a reading of the description of preferred embodiments which follows when taken in conjunction with the accompanying drawings.

### BRIEF DESCRIPTION OF THE DRAWINGS

FIG. 1 is an isometric view of the improved surgical instrument.

FIG. 2 is an exploded isometric view of the articulating tool assembly.

FIG. 3 is a side view of the articulating tool assembly.

FIG. 4 is a top view of the articulating tool assembly.

FIG. 5a is a cross sectional view of FIG. 3.

FIG. 5b is another cross sectional side view of the articulating tool assembly.

FIG. 6a is a cross sectional view of FIG. 4.

FIG. 6b is another cross sectional top view of the articulating tool assembly.

FIG. 7 shows one embodiment of the cabling in the improved surgical instrument.

FIG. 8 is an exploded isometric view of user interface.

FIG. 9 is an isometric view of the contents within the head casing of the user interface.

FIG. 10 is a top view of user interface partially exposing the contents within the head casing.

FIG. 11 is an isometric view of the user interface.

FIG. 12 is cross sectional view of the user interface.

### DETAILED DESCRIPTION

The present invention is an improvement of the instrument disclosed in Application Number PCT/US2011/027061 published as WO 2011/109640, the entirety of its contents is incorporated herein.

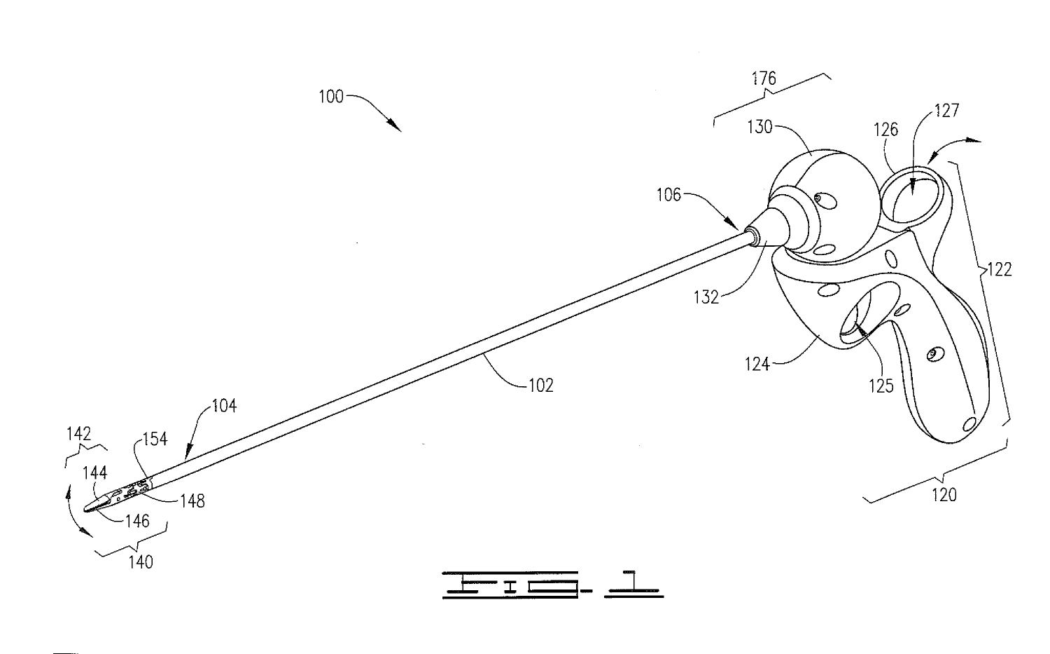

FIG. 1 depicts an isometric view of instrument 100. Instrument 100 may be manufactured from any suitable surgically safe materials, such as, but not limited to, stainless steel, aluminum, titanium, plastics, poly(methyl methacrylate), polytetrafluorethylene, composites, or some combination thereof. A sample instrument 100 suitable for teaching and demonstration purposes may use a lightweight, transparent material, such as a transparent thermoplastic, for example poly(methyl methacrylate) in place of the standard surgical material. Instrument 100 includes a user interface 120. Using user interface as a point of reference, instrument 100 includes elongate tubular member 102 having a distal end 104 and a proximal end 106. User interface 120 is coupled to proximal end 106. Instrument 100 also includes an articulating tool assembly 140 coupled to distal end 104. User interface 120 is operatively connected to articulating tool assembly 140 by at least one cabling member 108 extending through elongate tubular member 102. User interface 120 controls or manipulates articulating tool assembly 140 such that movement at user interface 120 is translated to articulating tool assembly 140 through cabling member 108. It is also through cabling member 108 that the user receives the tactile feedback of the movements. Some non-limiting examples of the ratio of movement at articulating tool assembly 140 to movement at user interface 120 may be any ratio suitable for the various surgical procedures including, a 1:1 ratio up to a 4:1 ratio and all ratios therebetween to accommodate for different types of articulating tool assemblies and procedures. For example, suitable ratios include: 1:1, 1.25:1, 1.5:1, 1.75:1, 2:1, 2.25:1, 2.5:1, 2.75:1, 3:1, 3.25:1, 3.5:1, 3.75:1, 4:1, and all ratios therebetween. The desired ratio may be specified at the time of manufacture and/or adjustable within instrument 100. The range of motion of articulating tool assembly 140 is comparable to that of the human wrist.

Instrument 100 provides one-handed operation and direct tactile sensations transmitted to the user during use. Since user input or movement of user interface 120 provides corresponding movement in the same direction at articulating tool assembly 140, instrument 100 is intuitive and easy to learn and operate. Instrument 100 will normally be used in a variety of fields where the distal end 104 and articulating tool assembly 140 will be separated from the user interface 120 by a barrier. Typically, articulating tool assembly 140 will be disposed within a cavity, for example but not limited to, an anatomical cavity (not depicted), with user interface 120 disposed external to the cavity. In a preferred embodiment, instrument 100 is used for performing open surgical procedures and endoscopic procedures such as laparoscopic and/or thorascopic surgeries on human subjects and veterinary subjects.

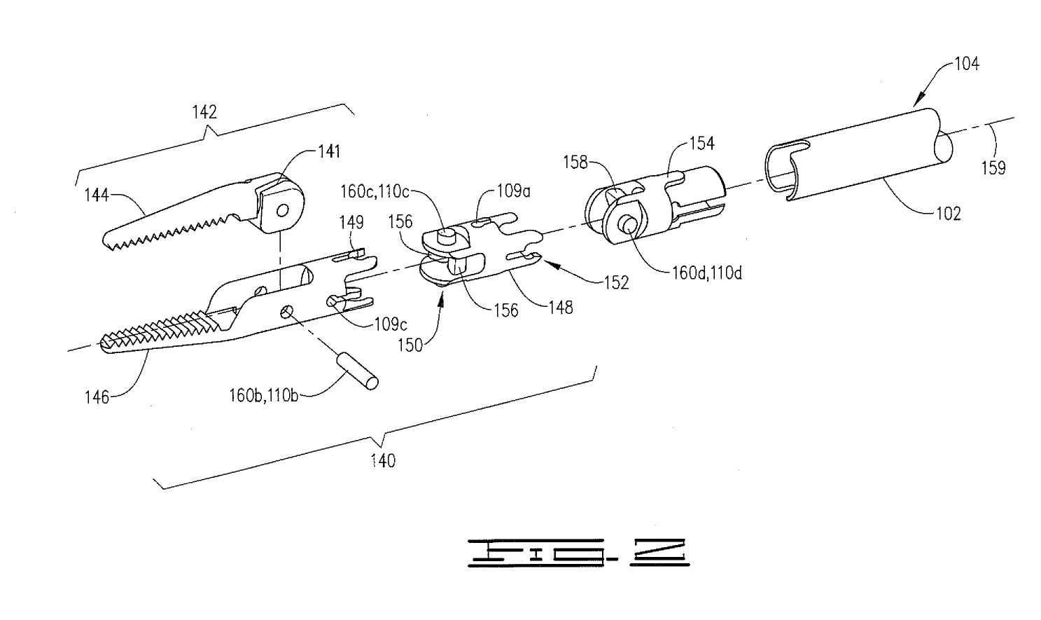

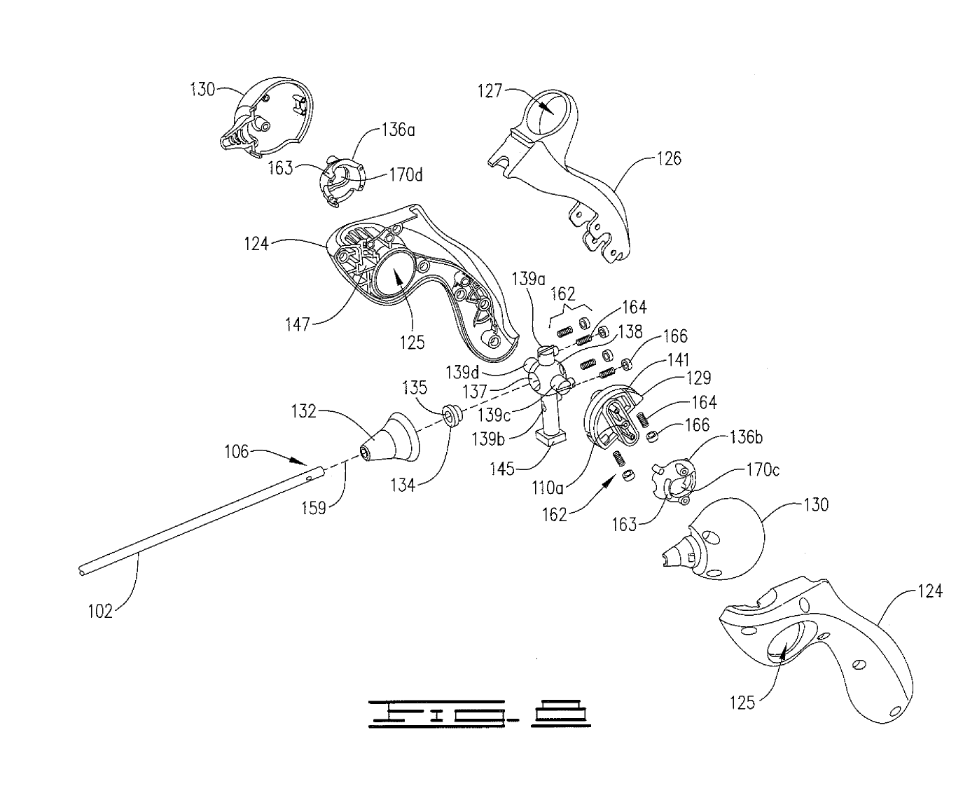

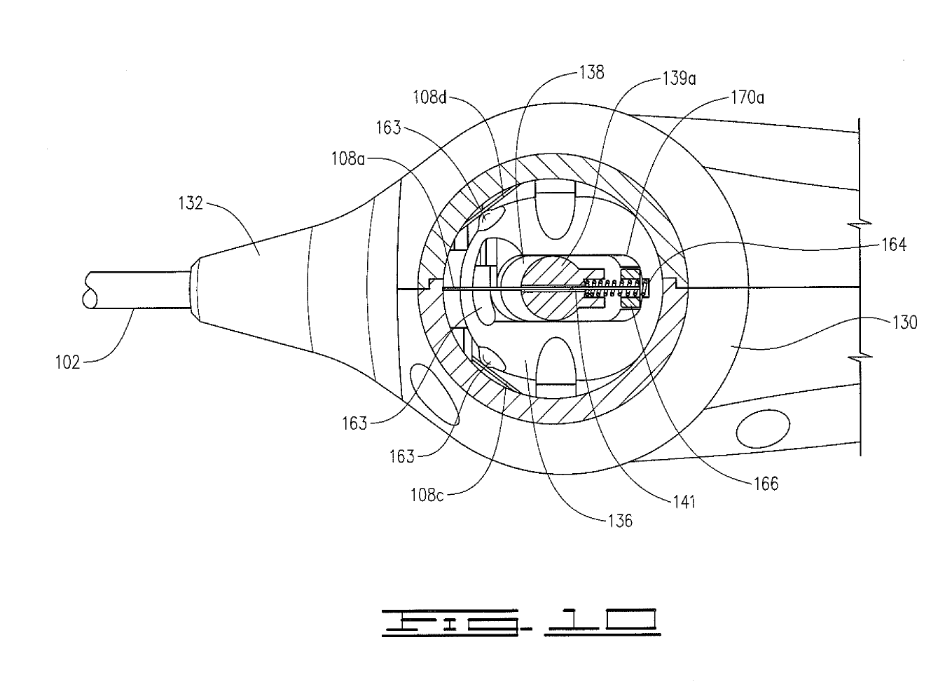

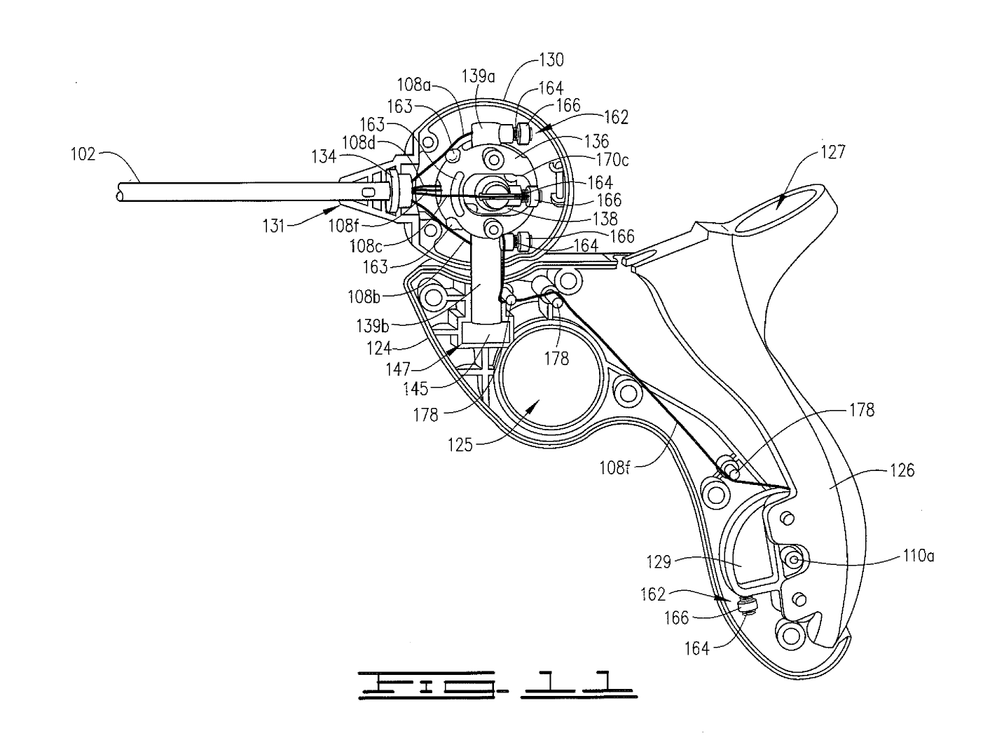

FIGS. 2 and 8 depict exploded views of the tool end of instrument 100 including articulating tool assemblies 140, and the user interface 120, respectively. User interface 120 as depicted in FIGS. 1 and 8-12 includes handle assembly 124 operatively coupled to ball and socket assembly 128, and a support assembly 176. Support assembly 176 includes head casing 130. In other embodiments, support assembly 176 also includes outer head cover 132 and guide member 134. Support assembly 176 secures ball and socket assembly 128 within head casing 130 as shown in FIGS. 10 and 11. As shown in FIG. 10, socket assembly 128 is secured to head casing 130 by securing mechanisms such as screws, pins, dowels, snaps, etc. For clarity of the drawings, securing mechanisms are not shown.

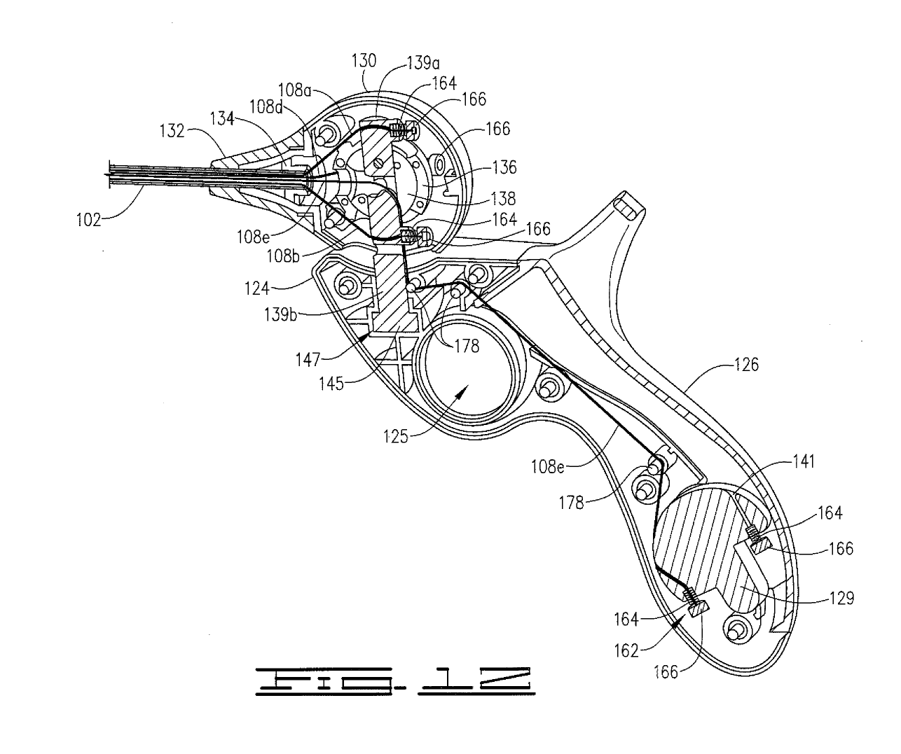

As depicted in FIGS. 8 and 11-12, user interface 120 provides an attachment site for securing elongate tubular member 102 to user interface 120. Elongate tubular member 102 may be secured to interface 120 by any convenient means including but not limited to a press fit within a bore of guide member 134 or threaded into a bore of guide member 134. Additionally, guide member 134 provides a passage 135 for cabling members 108 to enter elongate tubular member 102. As shown in the figures, head casing 130 has a tapered portion 131. Guide member 134 may be positioned at any convenient location within head casing 130. As shown in FIGS. 11 and 12, guide member 134 is positioned within head casing 130 where head casing 130 begins to taper. Preferably, guide member 134 is made from stainless steel and has an approximate outermost diameter ranging from about 5 mm to about 15 mm. In one embodiment the preferred outermost diameter of guide member 134 is about 11 mm. It should be appreciated that the preferred dimensions of instrument 100 will vary according to the type of surgical procedure and/or size of the patient. Elongate tubular member 102 is made from stainless steel and preferably has an outside diameter of about 5 mm to about 12 mm. In one embodiment, elongate tubular member 102 has a preferred outside diameter of about 5 mm. The length of elongate tubular member 102 ranges from about 304.8 mm to about 457.2 mm with a typical length of about 304.8 mm (12 in) depending upon the procedure to be performed.

In a preferred embodiment, ball and socket assembly 128 is supported by support assembly 176. Ball and socket assembly 128 is secured within head casing 130 as shown in FIG. 10. Head casing 130 is generally round in shape, but any other shape or casing capable securing ball and socket assembly 128 is suitable for the claimed invention. Handle assembly 122 is directly coupled to ball and socket assembly 128 by protruding arm 139b for direct transference of movement of handle assembly 122 to the articulating tool assembly 140 via cabling member 108.

As depicted in FIGS. 8-12, ball and socket assembly 128 is positioned within support assembly 176. Ball and socket assembly 128 includes ball 138, depicted in FIGS. 8, 9, 11 and 12. Ball 138 is generally spherical in shape. Ball 138 has a central bore 137 thereby allowing cabling members 108e and 108f to pass therethrough and into elongate tubular member 102. Ball 138 has top, bottom, left, and right protruding arms 139a, 139b, 139c, and 139d, respectively. In a preferred embodiment, protruding arms 139 are cylindrical in shape; however, arms 139 can be any suitable shape for coupling cabling member 108 to ball 138. Each protruding arm 139 provides an attachment point for one cabling member 108 (108a, 108b, 108c, and 108d). In one embodiment ball 138 has a diameter of about 15 mm to about 25 mm. In one embodiment, the diameter of ball 138 is about 18 mm-about 19 mm. Ball 138 is made surgically safe material. In one embodiment, ball 138 is made of stainless steel, in other embodiments ball 138 is made of polytetrafluorethylene.

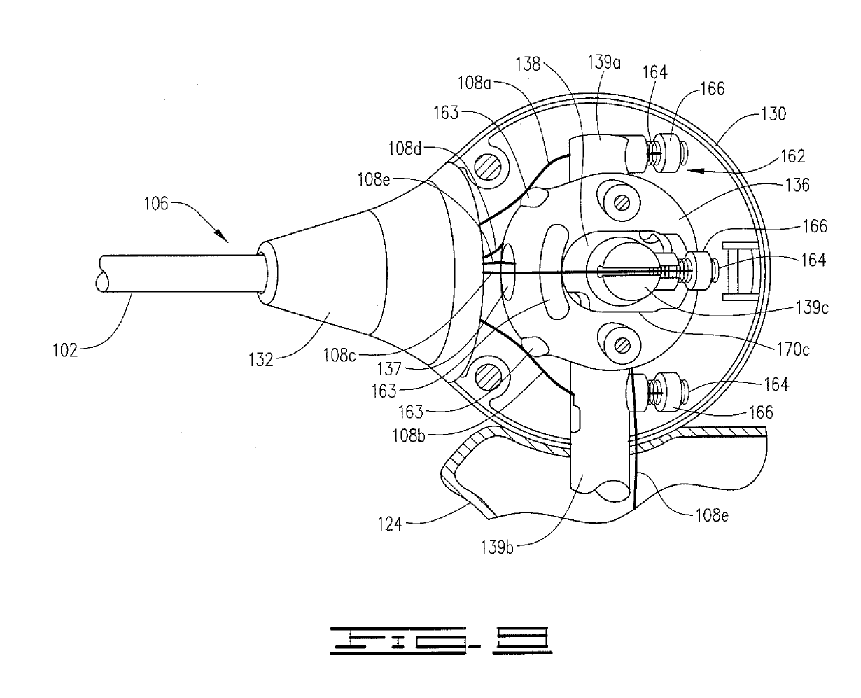

Cabling members 108 are made of a material that is strong and surgically safe. Suitable material includes, but is not limited to, 304 stainless steel nylon coated cable. Any similar component suitable for operatively coupling user interface 120 with articulating tool assembly 142 will perform satisfactorily in the current invention, such similar component can be used in, for example, non-surgical procedures such as training or demonstrative purposes. In the depicted embodiments, cabling members 108 are pre-tensioned cables each having swage balls 107 attached on the ends. Cabling members 108 may be directly attached to arms 139, including, but not limited to, tying, solding, or other suitable direct attachments. Alternatively, any suitable securing device known in the art, for example, bolts, pins, buttons, press-fitted pins, and screws or any similar component suitable for securing cabling member 108 to protruding arm 139 will perform satisfactorily in the current invention. In a preferred embodiment, cabling member 108 is secured to protruding arms 139 as shown in FIGS. 8-12 by spring tension assembly 162. Spring tension assembly 162 is carried by protruding arms 139. Spring tension assembly includes a spring 164 and a cable stopper 166. Spring 164 carries cable stopper 166. Cable member 108 is disposed within spring 164 of spring tension assembly 162 as shown in FIGS. 9-11. Cable stopper 166 has a recessed cavity, similar to cable termination points 109, to receive swage ball 107 thereby securing cable 108 to arm 139. Cable stopper 166 is made of surgically safe material, including, for example, stainless steel. Spring tension assembly 162 provides sufficient tension in cabling members 108 to compensate for minor cable length differences as ball 138 moves, to compensate for cable length tolerances, and to compensate for cable stretch or wear on cabling members during use of instrument 100. Protruding arms 139 also have guide grooves 141 as depicted in FIGS. 9-11 to guide cabling members 108.

Referring to FIGS. 8-12, ball 138 is disposed within a generally spherical cavity formed by socket members 136a, 136b of socket 136. Assembled socket 136 has openings 170 for protruding arms 139a, 139b, 139c, and 139d to extend through to define the range of movement for ball 138 and permit unobstructed movement of ball 138 within socket 136. This configuration provides the optimal range of movement for ball 138. As discussed above, this range of movement translates directly to articulating tool assembly 142.

In one embodiment shown in FIG. 10, top and bottom openings 170a, 170b (only one depicted) of socket 136 through which top and bottom protruding arms 139a, 139b project are generally elliptical in shape thereby restricting the range of motion of the top and bottom protruding arms 139a and 139b. Openings 170c, 170d through which left and right protruding arms 139c, 139d project are generally rectangular in shape thereby permitting a full range of left and right movement of arms 139c and 139d with respect to the location of the top and bottom protruding arms 139a, 139b.

As shown in FIGS. 9-12, socket 136 has elevated surfaces 163 to guide and support cabling members 108 as cabling members are channeled through passage 135 of guide member 134. The dimensions and material of socket 136 can vary per application; in one embodiment socket 136 is made of stainless steel, in other embodiments socket 136 is made of polytetrafluorethylene. As shown in FIG. 8, socket 136 is depicted as two pieces secured together via securing mechanisms (not shown) such as screws, pins, snaps, dowels, etc. Depending on the application, the dimensions of socket 136 can vary. Socket 136 may have outermost dimensions of about 25 mm-about 30 mm long, about 25 mm-about 30 mm wide, and about 25 mm-about 30 mm high. As shown in FIG. 10, socket 136 is secured to head casing 130 by securing mechanisms (not shown) such as screws, pins, dowels, etc.

As shown in FIGS. 8-12, outer head cover 132 covers the tapered portion 131 of head casing 130. Outer head cover 132 can be secured to head casing 130 by any suitable mechanism for securing outer head cover 132 to head casing 130. In one embodiment, outer head cover 132 is snapped into place against head casing 130. In another embodiment, outer head cover 132 is pushed against head casing 130 over the tapered portion 131 and twisted (rotated about longitudinal axis 159) into a final position as the tapered portion contains a thread for receiving and coupling with outer head cover 132. Such a configuration increases strength of the connection point and improves clamping on elongate tubular member 102.

In a preferred embodiment, handle assembly 122 includes grip member 124 and lever member 126 pivotally coupled to grip member 124 at pivot point 110a. Preferably, grip member 124 and lever member 126 are ergonomically designed to comfortably fit within the surgeon's hand. Grip member 124 is about 100 mm-150 mm tall, about 30 mm-50 mm wide at its widest point, and 100 mm-130 mm long. Grip member is made of materials suitable for use in surgery. In one embodiment, grip member 124 is made of polytetrafluorethylene. Lever member 126 is made of the same materials of grip member 124; in alternative embodiments, lever member 126 and grip member 124 are different materials.

Lever member 126 actuates the opening and closing of tip assembly 142. Movement of lever member 126 clockwise and counterclockwise about pivot point 110a causes tip assembly 142 to move between fully closed and fully open, and all positions therebetween.

Grip member 124 is directly coupled to ball 138 at bottom protruding arm 139b, thereby directly translating movement of handle assembly 122 to ball and socket assembly 128. As shown in FIGS. 8, 11 and 12, protruding arm 139b extends down into the handle assembly 122 and has a retention mechanism 145 which is positioned within a complimentary-shaped cavity 147 for receiving retention mechanism 145. Retention mechanism may be made of any suitable low friction material and any suitable shape that will enable the retention mechanism to fit snugly within a complimentary-shaped cavity 147. In the depicted embodiment shown in FIGS. 8 and 11, retention mechanism 145 is shaped as a rectangular prism and the complimentary-shaped cavity 147 is shaped to receive a rectangular prism. Suitable low friction materials for retention mechanism provide the user with a smooth or more fluid-like tactile sensation. Use of high friction materials will impart a sticking sensation and/or erratic movements at the user interface 120 precluding the desired tactile sensations.

User interface 120 is made of surgically safe materials, in one embodiment user interface utilizes stainless steel, aluminum, titanium, plastics, composites, and combinations thereof. The length of user interface 120 is approximately 120 mm to about 175 mm with a preferred length of about 146 mm and a height of about 110 mm to about 200 mm with a preferred height of about 150 mm and width ranging from about 30 mm to about 70 mm and a preferred width of approximately 46 mm. Again, the dimensions of instrument 100 will vary per application.

In a preferred embodiment, articulating tool assembly 140 includes interchangeable tip assembly 142 and dual hinge member 148. Depending on the intended use of instrument 100, tip assembly 142 may be selected from any of the following non-limiting examples: graspers, dissectors, scissor and blade tip assemblies. To provide cauterization capabilities, one of the cabling members 108 or an additional wire (not shown) provides an electrical current to the distal end 143 of tip assembly 142. For cauterization purposes, the material forming the distal end 143 of tip assembly 142 responds to the electrical current by producing heat sufficient to cauterize tissue. Except with regard to the distal end 143 of tip assembly 142, the remaining portion of instrument 100, which comes into contact with tissue, will be insulated to protect the surrounding tissue from injury.

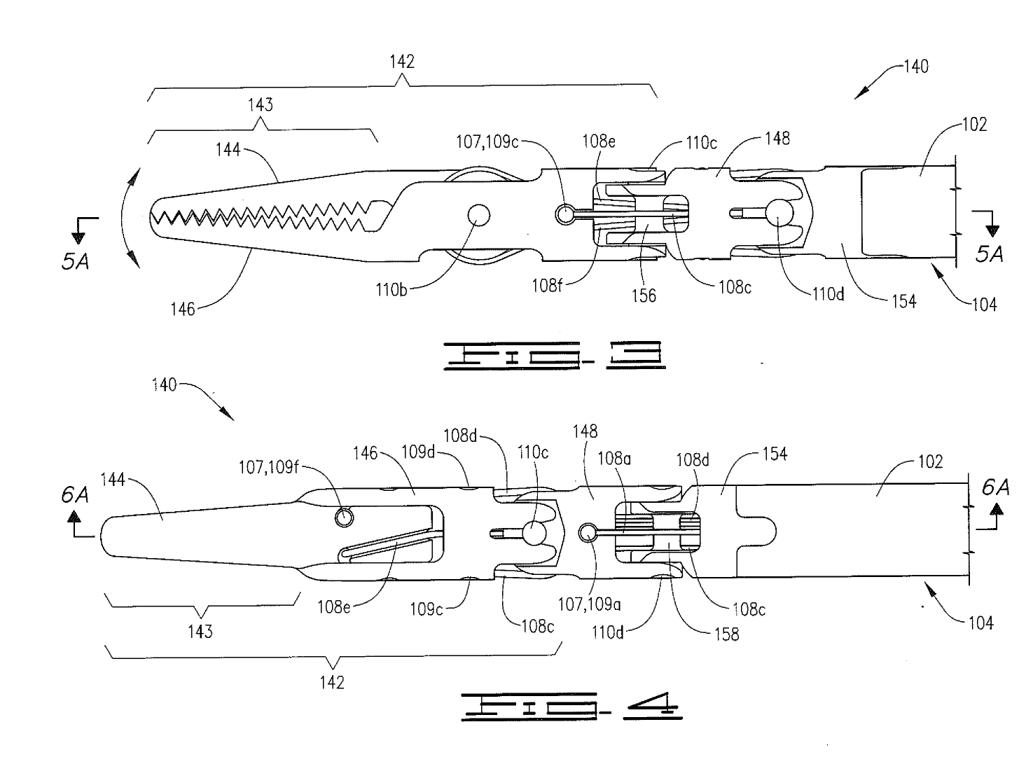

FIGS. 1-6 depict a grasper embodiment of tip assembly 142. As shown, tip assembly 142 includes a moving member 144 pivotally connected to base member 146 at pivot point 110b with pivot fastener 160b. Pivot fasteners 160 can be any fastener, suitable for operatively connecting the various components which are pivotally connected. Fasteners such as snap fasteners, dowels, rods, bolts, pins, buttons, press-fitted pins, screws, or any similar component will perform satisfactorily in the current invention. Modified versions of the exemplary fasteners include a fastener having a central bore passing perpendicularly therethough along either the horizontal or vertical axis of the fastener will perform satisfactorily in the current invention. As shown in FIG. 2, pivot fastener 160b is a smooth type fastener, such as, but not limited to, a dowel, rod, pin, press-fitted pin, etc. and pivot fasteners 160c and 160d are capable of being fastened to another object via a snapping mechanism.

To open tip assembly 142, the user will move actuating lever member 126 clockwise with respect to pivot point 110a when viewing instrument 100 as depicted in FIGS. 1 and 11. Conversely, to close tip assembly 142, the user will move actuating lever member 126 counterclockwise with respect to pivot point 110a when viewing instrument 100 as depicted in FIGS. 1 and 11. As shown in FIG. 1, the neutral position for tip assembly 142 is closed. In one embodiment, as shown in FIGS. 11 and 12, a user's hand will grasp handle assembly 122 with the user's thumb positioned within thumb hole 127 of the lever member 126. Depending on the user's comfort, the user can optionally place one or more fingers within finger hole 125 on grip member 124. To open tip assembly 142, the user will press his/her thumb against the structure of lever member 126 defining thumb hole 127 causing lever member 126 to rotate clockwise about pivot point 110a. The motion the user will make to open tip assembly is akin to making a “thumbs up” or a hitchhiking thumb hand gesture.

In the embodiment where instrument 100 is equipped with cautery capabilities, a control mechanism (not depicted), such as a button, switch, plug, or other actuation device for initiating and terminating the cautery feature is located on the grip member 124 near finger hole 125. The control mechanism for the cautery feature may also be elsewhere on instrument 100.

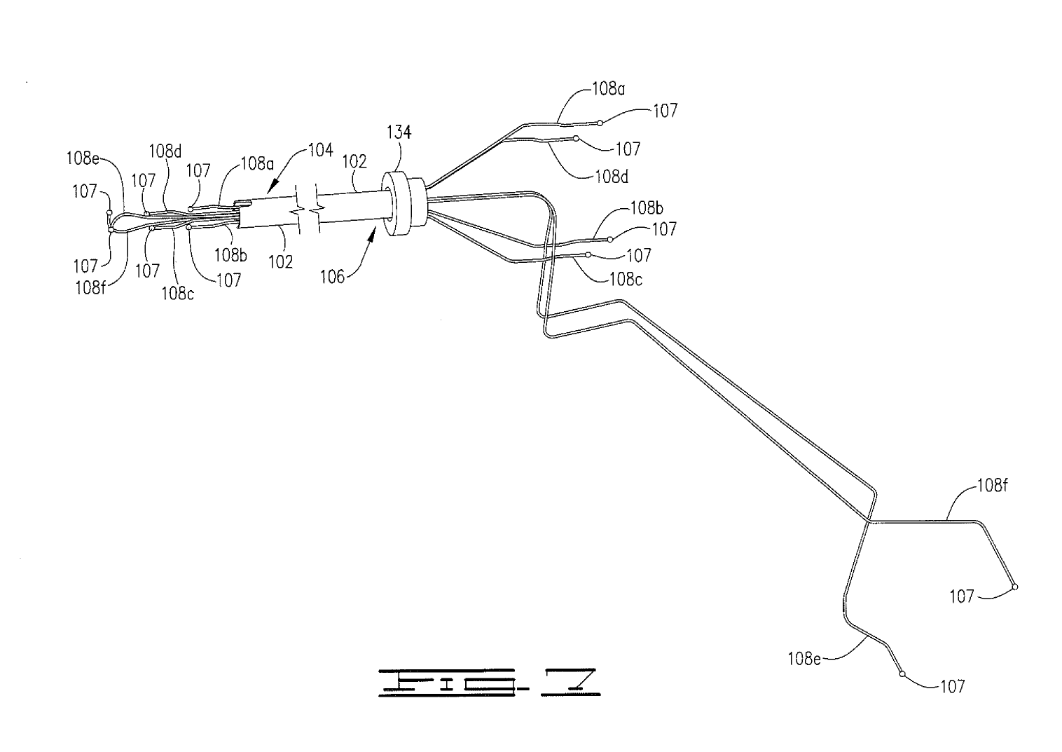

In a preferred embodiment, lever member 126 and tip assembly 142 are operatively connected via cabling member 108. A single cabling member is suitable for operatively connecting lever member 126 and tip assembly 142. A preferred embodiment is depicted in FIGS. 7, 11 and 12 with two cabling members 108e and 108f operatively connecting lever member 126 and tip assembly 142. Use of two cabling members to open tip assembly 142 provides for sufficient tension for opening, as well as provides the user with a more fluid/smooth (e.g. not jerk-like) tactile feedback, when opening and closing tip assembly 142.

As shown in FIGS. 7, 8, and 11-12, cabling members 108e and 108f are positioned on opposite sides of lever pivot member 129. Lever pivot member 129 has a circumferential groove and cable guide grooves 141 for receiving and guiding cabling members 108e and 108f along the circumference of lever pivot member 129 during actuation. Referring to FIG. 7 in combination with FIGS. 11 and 12, cabling members 108e and 108f extend up through grip member 124 and through a central bore 137 in ball 138 and through elongate tubular member 102 (along the longitudinal axis 159). Referring now to FIG. 7 in combination with FIGS. 2-6b, cabling members 108e and 108f are positioned within circumferential guide grooves 141 on moving member 144 and are secured to cable termination points 109e and 109f via swaged balls 107. Thus, actuating lever member 126 causes moving member 144 of tip assembly 142 to move in relation to base member 146 about pivot point 110b.

Spring tension assembly 162 provides the securing mechanism of cabling members 108e and 108f to lever pivot member 129. For brevity, the description of spring tension assembly 162 described will not be repeated here.

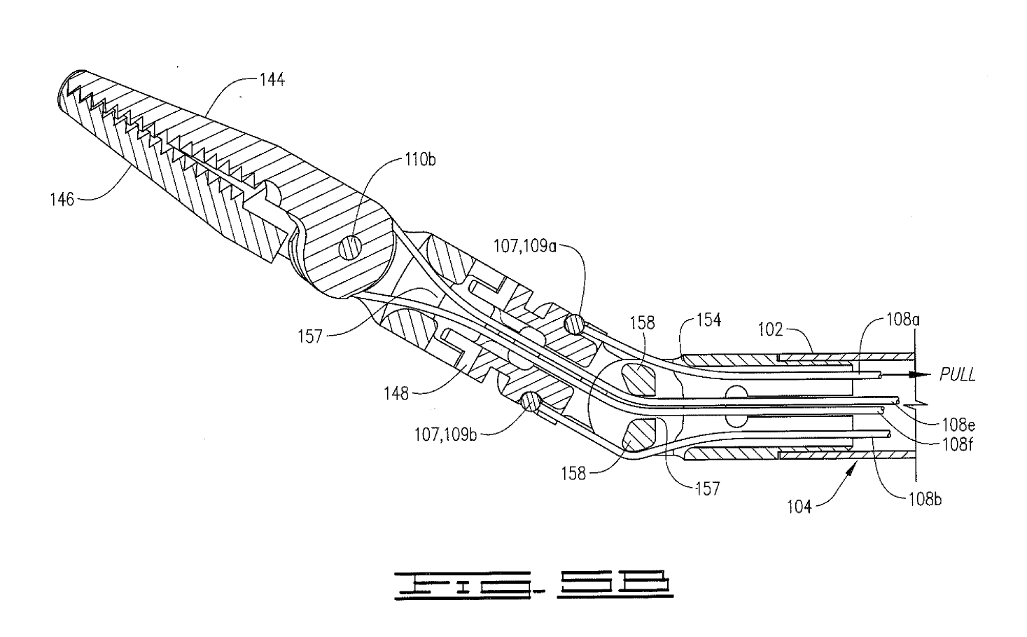

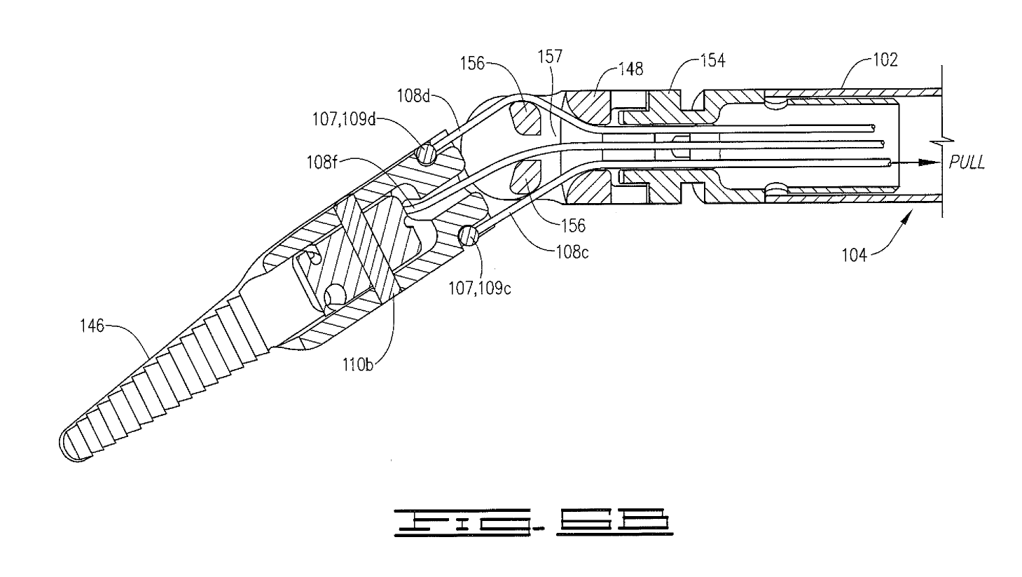

Precise and controlled movements during surgical procedures are paramount to ensuring the patient's safety and reducing unnecessary injuries and/or death. One embodiment of routing for cabling members 108 is shown in the figures. To provide precise, smooth, non jerk-like, and/or controlled movements, cabling members 108 extend as close as possible through the longitudinal axis 159 of elongate tubular member 102. As shown in FIGS. 5a, 5b, 6a, and 6b, tip assembly 142, dual hinge member 148, and elongate guide member 154 are configured to align the appropriate cabling members through a central passageway 157 defined by at least one cable alignment surface 156, 158, through cable guide grooves 141 (e.g. within tip assembly 142), and/or position cables along cable alignment surfaces 156, 158. Such cabling configuration precludes entanglement with adjacent cabling members 108, provides for sufficient tension and/or pull on the respective cabling members during operation thereby preventing the instrument from becoming “locked” and/or causing errant or unexpected movements in the articulating tool assembly 140.

Turning now to FIGS. 2-6b, in one embodiment articulating tool assembly 140 also includes elongate guide member 154. As depicted in the FIG. 2, elongate guide member 154 is depicted as a separate piece that connects to elongate tubular member 102 and dual hinge member 148. In another embodiment (not depicted), elongate guide member 154 and elongate tubular member 102 are an integral single component.

Referring to FIGS. 2-6b, dual hinge member 148 has a first side 150 and a second side 152. First side 150 of dual hinge member 148 is an outwardly projecting surface as shown in FIG. 2. Dual hinge member is generally hollow and has a central passageway 157 passing therethrough to allow cabling members 108 to be routed through the center of dual hinge member. The central passageway 157 is defined by cable alignment surfaces 158 of dual hinge member 148. Tip assembly 142 and elongate guide member 154 are pivotally coupled to dual hinge member 148 at pivot points 110c and 110d, respectively. The depicted coupling provides a snap pivot point. Any type of pivot mechanism is suitable so long as the desired effect is achieved and appropriate tension on cabling members 108 is achieved. As shown in FIGS. 2-4, tip assembly 142, specifically base member 146 has an outwardly projecting flexible tab 149 capable of receiving pivot fastener 160c. As shown in FIG. 2, pivot fastener 160c projects out (up and down) from the first side 150 of dual hinge member 148. Pivot fastener 160c is an outwardly projecting connecting surface capable of being received by outwardly projecting tab 149 of base member 146. As used herein the outwardly projecting flexible tab is a component that flexes around an object or post and returns to its original position thereby connecting one component to another.

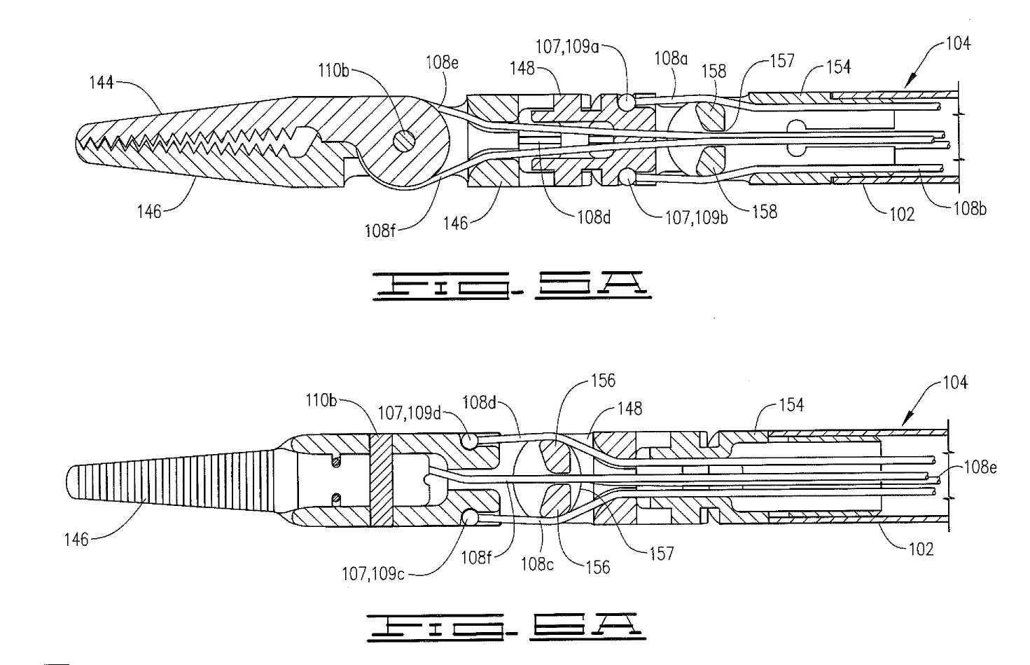

As depicted in FIG. 2, the operative coupling of base member 146 with dual hinge member 148 permits lateral movement, i.e. left and right movement, of tip assembly 142 with respect to the longitudinal axis 159 of elongate tubular member 102 as depicted in FIG. 2. For example, cabling members 108c and 108d, shown in FIGS. 4, 6a, 6b, 7, and 9-12 operatively connect to the left and right protruding arms 139c, 139d, respectively, of ball 138. From the perspective of looking down instrument 100 from user interface 120, when a user moves handle assembly 122, for example, twisting or rotating clockwise or counterclockwise, or right or left with respect to longitudinal axis 159 of elongate tubular member 102, the tip assembly 142 laterally pivots about point 110c.

As shown in FIGS. 5a, 5b, 6a, and 6b cable alignment surface 158 of elongate guide member 154 and cable alignment surface 156 of dual hinge member 148 define a central passageway 157 through elongate guide member 154 and dual hinge member 148, respectively. Central passageway 157 guides passive cabling members through pivot points 110d and 110c, to prevent any change in length of the cable, and also guides active cabling members away from the pivot point to prevent active cabling members from crossing over longitudinal axis 159 and causing tip assembly 142 to “lock up” or become stuck in a position. If instrument 100 becomes “locked,” instrument 100 is unable to return to a neutral position (as shown in FIG. 1) or capable of being moved to another position, or capable of having full range of motion and/or smooth, tactile, non jerk-like movements.

As used herein passive cabling members are cabling members passing through a pivot point, active cabling members are cabling members pulled to bend at a specific pivot point. As will be described below, whether a cabling member 108 is active or passive is determined by the movement imparted to articulating tool assembly 140 by user interface 120. For example, referring to FIGS. 5a and 5b, cabling members 108a, 108b, 108e, and 108f are shown. Due to the cross section of FIGS. 5a and 5b, cabling members 108c and 108d are not depicted. As shown in FIGS. 5a and 5b, cabling members 108e and 108f are considered passive cabling members as they are guided through pivot point 110d. Although not depicted, cabling members 108c and 108d are also passive cabling members through pivot point 110d. As shown in FIG. 5b, as the tool end is manipulated, passive cabling members are guided along the cable alignment surface 156 to dual hinge member 148 and tip assembly 142 in such a manner as to prevent any change in length in the cabling members thereby preventing any undue tension on the passive cable members and preventing any errant movements in the tip assembly 142. In FIG. 5b, active cabling members are cabling members 108a and 108b. Cabling members 108a and 108b are directed away from the pivot axis and guided toward the exterior of cable alignment surface 156 and secured to dual hinge member 148 at cable termination points 109a and 109b. As shown in FIG. 5b, cabling member 108a is being pulled by user interface 120 thereby causing the articulating tool assembly to move up with respect to longitudinal axis 159 of elongate tubular member 102.

Referring to FIGS. 5-6, cabling members 108c, 108d, 108e, and 108f continue onward from elongate guide member 154 until they arrive at their respective cable termination points 109. In FIGS. 6a and 6b, dual hinge member 148 also has cable alignment surface 158. As shown in FIG. 6b, cabling members 108e and 108f are still passive cabling members and are guided to tip assembly 142 to their respective cable termination points 108e and 108f by central passage 157. In FIG. 6b, cabling members 108c and 108d are active cabling members and are guided away from passing though the center of pivot point 110c. As shown in FIG. 6b, cabling members 108c and 108d are guided along the exterior walls of cable alignment surface 158 to cable termination points 109c and 109d, respectively. As shown in FIG. 6b, cabling member 108c is being pulled by user interface 120 thereby causing articulating tool assembly 140 to move left with respect to longitudinal axis 159 of elongate guide member 102.

Preferably, cable alignment surfaces 156 and 158 are smooth surfaces without any sharp edges in order to reduce friction and wear on the cabling members. Cable alignment surfaces can be beveled, rounded, or any other contoured surface suitable for guiding active and passive cabling members will perform satisfactorily in the current invention.

At the tool end, cabling members 108 are received within cable termination points 109. Other ways to secure the terminated end of the cable are envisioned, and any method capable of maintaining the desired tension for proper operation of instrument 100 will perform satisfactorily in the current invention. For example, cabling members may be secured to the termination point 109 via adhesive, tying, and/or by use of nuts and other fasteners, or press fitted. As shown in FIGS. 1-7, cabling members 108 have a swaged ball 107 attached to the ends of cabling members 108. In the handle assembly, swaged ball 107 is received by cable stopper 166.

Pivot point 110d controls vertical movement, i.e. up and down movement, with respect to the longitudinal axis 159 of elongate tubular member 102. As depicted in FIGS. 2-4, elongate guide member 154 is pivotally connected to the second side 152 of dual hinge member 148. Similar to the base member 146, the second side 152 of dual hinge member 148 is an outwardly projecting flexible tab capable of receiving pivot fastener 160d of elongate guide member 154. The outwardly projecting flexible tab is depicted in FIG. 2 running along longitudinal axis 159. Like the connection between dual hinge member 148 and tip assembly 142, the connection between elongate guide member 154 and dual hinge member 148 is a snap pivot. Elongate guide member 154 connects to dual hinge member 148 by an outwardly extending connecting surface 160d as shown in FIG. 2. As shown in FIG. 2, the outwardly projecting connecting surface 160d is capable of being received by the outwardly projecting tab 152 of dual hinge member 148.

For example, cabling members 108a and 108b, shown in FIGS. 4, 6a, 6b, 7, and 9-12, operatively connect the top and bottom protruding arms 139a, 139b, respectively, of ball 138 to dual hinge member 154. As discussed above, the configuration of openings 170a, 170b, 170c, and 170d define the limits of movement for protruding arms 139. Thus, operation of handle assembly 122, for example forwards or backwards along longitudinal axis 159 of elongate tubular member 102, the tip assembly 142 vertically pivots about point 110d. Such manipulation translates down cabling members 108a and 108b causing dual hinge member 148 to vertically pivot about point 110d. As already discussed, socket 136 has openings 170 to allow for protruding arms 139 to move unobstructed within a defined area. Vertical movement occurs as a result of the position of top and bottom protruding arms 139a and 139b, respectively.

It should be appreciated that the orientation of the outwardly extending tab 149 of base member 146, the first side 156 of dual hinge member 148, the second side of dual hinge member 152, and elongate guide member 154 are not limited to configuration described above or shown in the drawings. For example, pivot points 110c and 110d can provide vertical and horizontal movements, respectively. Other arrangements may be dictated by the nature of tip assembly 142 and the intended use instrument 100.

As shown in FIGS. 1-4, elongate guide member 154 is optionally coupled to elongate tubular member 102 by being slideably positioned within elongate tubular member 102. When assembled elongate guide member 154 is flush with elongate tubular member 102. In other embodiments elongate guide member 154 and elongate tubular member are a single integral component.

Instrument 100 is not limited to the dimensions and types of material used and configurations described above. Such characteristics of instrument 100 will vary depending on the application or use, for example, the type of surgical procedure, e.g. human or veterinary surgical procedures; size of patient or subject; use for training such as, but not limited to, use on mannequins or cadavers; use for demonstrative purposes in various environments such as: commercial settings like trade shows; medical offices; academic settings; or private settings. It should be appreciated that any similar component suitable for satisfactorily performing the function of the corresponding component can be used in the current invention.

Thus, the present invention is well adapted to carry out the objects and attain the ends and advantages mentioned above as well as those inherent therein. While certain embodiments of the invention have been described for the purpose of this disclosure, numerous changes in the construction and arrangement of parts and the performance of steps can be made by those skilled in the art, which changes are encompassed within the scope and spirit of this invention defined by the appended claims.

What is claimed is:

1. An instrument comprising:
an elongate tubular member having a distal end and a proximal end;
an articulating tool assembly coupled to said distal end, wherein said articulating tool assembly includes a tip assembly, and a dual hinge member having a first side and a second side, wherein said tip assembly is pivotally attached to said first side of said dual hinge member, wherein said dual hinge member includes at least one cable alignment surface therein, said dual hinge member having a central passageway passing therethrough;
a user interface coupled to said proximal end, wherein said user interface includes a ball and socket assembly, said ball and socket assembly includes a socket having openings and a ball having protruding arms, wherein said socket carries said ball and said protruding arms project from said openings of said socket, wherein said protruding arms carry a spring tension assembly;
at least one cabling member extending through said elongate tubular member, said at least one cabling member operatively connecting said user interface and said articulating tool assembly.

2. The instrument of claim 1, wherein said operative connection between said user interface and said articulating tool assembly provides corresponding movement such that movement at said articulating tool assembly corresponds to movement at said user interface.

3. The instrument of claim 2, wherein a ratio of movement of said articulating tool assembly to movement of said user interface is a three to one ratio.

4. The instrument of claim 1, wherein said spring tension assembly including a spring having a first end and a second end, and a cable stopper, wherein said first end of said spring is carried by said protruding arms, and wherein said cable stopper carries said second end of said spring.

5. The instrument of claim 1, wherein said at least one cable alignment surface prevents entanglement of said at least one cabling member and provides sufficient alignment and/or tension on said at least one cabling member thereby providing said instrument with precise movements and preventing unwanted movements in said articulating tool assembly.

6. The instrument of claim 2, wherein the user interface further includes a handle assembly operatively connected to said ball and socket assembly, and a support assembly supporting said ball and socket assembly, wherein said elongate tubular member is secured to said support assembly.

7. The instrument of claim 6, wherein said support assembly includes a head casing, wherein said elongate tubular member is secured to said support assembly by an outer head cover and a guide member, wherein said elongate tubular member is connected to said guide member within said head casing, and wherein said outer head cover carries said elongate tubular member.

8. The instrument of claim 2, wherein said articulating tool assembly further includes an elongate guide member coupled to said elongate tubular member, wherein said elongate guide member has a central passageway therethrough.

9. The instrument of claim 8, wherein said elongate guide member has an outwardly extending surface pivotally coupled to said second side of said dual hinge member.

10. The instrument of claim 9, wherein said elongate guide member has at least one cable alignment surface, wherein said at least one cable alignment surface prevents entanglement of said at least one cabling member and provides sufficient alignment and/or tension on said at least one cabling member thereby providing said instrument with precise movements and preventing unwanted movements in said articulating tool assembly.

11. The instrument of claim 10, wherein said second side of said dual hinge member is an outwardly projecting tab.

12. The instrument of claim 8, wherein said elongate tubular member and said elongate guide member are an integral component.

13. The instrument of claim 12, wherein said integral component has an outwardly extending surface pivotally coupled to said second side of said dual hinge member.

14. The instrument of claim 6, wherein the handle assembly includes a grip member, and a lever member pivotally coupled to the grip member, wherein actuation of the lever member controls opening and closing of said tip assembly.

15. The instrument of claim 14, wherein said tip assembly is positioned at any location between, and including, fully closed and fully opened by actuation of said lever member.

16. The instrument of claim 15, wherein said tip assembly responds to an electrical current by producing heat sufficient to cauterize tissue.

17. The instrument of claim 14, wherein said dual hinge member provides lateral and vertical movement of the tip assembly.

18. The instrument of claim 1, wherein said openings define the range of motion for said protruding arms.

19. A surgical instrument comprising:
an elongate tubular member having a distal end and a proximal end, wherein said elongate tubular member has a length sufficient to permit position of the distal end within an anatomical cavity while the proximal remains external to the anatomical cavity;
an articulating tool assembly coupled to said distal end of said elongate tubular member, the articulating tool assembly including a dual hinge member having a first side and a second side, and a tip assembly, said tip assembly pivotally connected to said first side of said dual hinge member, wherein said tip assembly includes a base member having a outwardly projecting tab pivotally connected to said first side of said dual hinge member, and a moving member pivotally connected to said base member, and wherein said dual hinge member has a central passageway therethrough;
a user interface coupled to the proximal end of said elongate tubular member and operatively connected to said articulating tool assembly, wherein movements made at the user interface moves the articulating tool assembly, wherein a ratio of movement between said articulating tool assembly to said user interface is three to one, wherein the user interface includes a handle assembly, a ball and socket assembly operatively connected to said handle assembly, and a support assembly supporting said ball and socket assembly, wherein said proximal end of the elongate tubular member secured to said support assembly, wherein the ball and socket assembly includes a ball having projecting arms disposed within a socket having openings to allow the projecting arms of the ball to move within the socket, wherein the handle assembly includes a grip member and a lever member operably connected to the grip member, wherein the grip member is operatively connected to one of the projecting arms of the ball, wherein said lever member is operatively connected to said moving member of said tip assembly thereby permitting the opening and closing of said tip assembly, wherein said support assembly includes a head casing supporting said ball and socket assembly;
and at least one cabling member operatively connecting said user interface and said articulating tool assembly, wherein said at least one cabling member is secured to said protruding arms of said ball by a spring tension assembly, said spring tension assembly carrying said at least one cabling member, wherein said spring tension assembly includes a spring having a first side and a second side, and a cable stopper, said protruding arms carrying said first side of said spring, and wherein said cable stopper carries said second side of said spring,
wherein said dual hinge member includes at least one cable alignment surface, wherein said at least one cable alignment surface prevents entanglement of said at least one cabling member and provides sufficient alignment and/or tension on said at least one cabling member thereby providing said instrument with precise movements and preventing unwanted movements in said articulating tool assembly.

20. The instrument of claim 19, wherein said grip member and said lever member are pivotally coupled, and wherein actuation of said lever member controls opening and closing of said tip assembly.

21. The instrument of claim 20, wherein said tip assembly is positioned at any location between, and including, fully closed and fully opened by actuation of said lever member.

22. The instrument of claim 21, wherein said tip assembly responds to an electrical current by producing heat sufficient to cauterize tissue.

23. The instrument of claim 20, wherein said dual hinge member provides lateral and vertical movement to the tip assembly.

24. The instrument of claim 19, wherein said articulating tool assembly further includes an elongate guide member coupled to said elongate tubular member, wherein said elongate guide member has at least one cable alignment surface, wherein said elongate guide member has a central passageway therethrough, and wherein said at least one cable alignment surface prevents entanglement of said at least one cabling member and provides sufficient alignment and/or tension on said at least one cabling member thereby providing said instrument with precise movements and preventing unwanted movements in said articulating tool assembly.

25. The instrument of claim 24, wherein said elongate guide member has an outwardly extending surface pivotally coupled to said second side of said dual hinge member.

26. The instrument of claim 25, wherein said second side of said dual hinge member is an outwardly projecting tab.

27. The instrument of claim 24, wherein said elongate tubular member and said elongate guide member are an integral component.

28. The instrument of claim 27, wherein said integral component has an outwardly extending surface pivotally coupled to said second side of said dual hinge member.

29. The instrument of claim 19, wherein said elongate tubular member is secured to said support assembly by an outer head cover and a guide member, wherein said elongate tubular member is connected to said guide member within said head casing and wherein said outer head cover surrounds said elongate tubular member.

30. An improved articulating tool assembly suitable for providing precise control of a surgical instrument and preventing unwanted movements, said articulating tool assembly comprising:
a tip assembly having a base member and a moving member pivotally coupled to said base member, wherein said base member has an outwardly projecting tab, wherein said tip assembly is configured to be positioned at fully opened and fully closed and all positions therebetween, wherein said tip assembly responds to an electrical current by producing heat sufficient to cauterize tissue;
a dual hinge member pivotally coupled to said tip assembly by said outwardly projecting tab of said base member of said tip assembly, wherein said dual hinge member has a first side and a second side, wherein said dual hinge member has a central passageway therethrough, wherein said dual hinge member provides lateral and vertical movement of the tip assembly; wherein said first side of said dual hinge member has an outwardly projecting connecting surface capable of being received within said outwardly projecting tab of said base member, wherein said second side of said dual hinge member is an outwardly projecting tab, and wherein said dual hinge member has at least one cable alignment surface, wherein said at least one cable alignment surface provides precise movements of and prevents unwanted movements of said articulating tool assembly; and
an elongate guide member pivotally coupled to said second side of said dual hinge member, wherein said elongate guide member has a passageway therethough, wherein said elongate guide member has an outwardly extending connecting surface capable of being received within said outwardly projecting tab of said dual hinge member, wherein said elongate guide member wherein said elongate guide member has at least one cable alignment surface, and wherein said at least one cable alignment surface provides precise movements of and prevents unwanted movements of said articulating tool assembly.

31. An improved articulating tool assembly suitable for providing precise control of a surgical instrument and preventing unwanted movements, said articulating tool assembly comprising:
a tip assembly having a base member and a moving member pivotally coupled to said base member, and
a dual hinge member pivotally coupled to said tip assembly, wherein said dual hinge member has a first side and a second side, wherein said dual hinge member has a central passageway therethrough, and wherein said dual hinge member has at least one cable alignment surface, wherein said at least one cable alignment surface provides precise movements of and prevents unwanted movements of said articulating tool assembly.

32. The articulating tool assembly of claim 31, wherein said tip assembly is configured to be positioned at fully opened and fully closed and all positions therebetween.

33. The articulating tool assembly of claim 31, wherein said tip assembly responds to an electrical current by producing heat sufficient to cauterize tissue.

34. The articulating tool assembly of claim 31, wherein said base member has an outwardly projecting tab, and wherein said first side of said dual hinge member has an outwardly projecting connecting surface capable of being received within said outwardly projecting tab of said base member.

35. The articulating tool assembly of claim 31, wherein said dual hinge member provides lateral and vertical movement of the tip assembly.

36. The articulating tool assembly of claim 31, further comprising:
an elongate guide member pivotally coupled to said second side of said dual hinge member, wherein said elongate guide member has a passageway therethough.

37. The articulating tool assembly of claim 36, wherein said second side of said dual hinge member is an outwardly projecting tab and wherein said elongate guide member has an outwardly extending connecting surface capable of being received within said outwardly projecting tab of said dual hinge member.

38. The articulating tool assembly of claim 36, wherein said elongate guide member wherein said elongate guide member has at least one cable alignment surface, and wherein said at least one cable alignment surface provides precise movements of and prevents unwanted movements of said articulating tool assembly.
